(19) United States
(12) Patent Application Publication
(10) Pub. No.: US 2011/0028430 A1
(43) Pub. Date: Feb. 3, 2011

(54) INJECTABLE VISCOUS MEDICINAL PREPARATION COMPRISING ETHANOL AND AN X-RAY OPAQUE FAT-SOLUBLE COMPOUND

(76) Inventors: Jacques Theron, Fleury Sur Orne (FR); Anne Dompmartin, Caen (FR); Daniel Labbe, Caen (FR)

Correspondence Address: YOUNG & THOMPSON, 209 Madison Street, Suite 500, Alexandria, VA, 22314, US

(21) Appl. No.: 12/876,474

(22) Filed: Sep. 7, 2010

Related U.S. Application Data
(62) Division of application No. 12/088,704, filed on Jul. 9, 2008, now abandoned.

(30) Foreign Application Priority Data
Sep. 30, 2005 (FR) 05/09978

(51) Int. Cl. A61K 31/717; A61P 43/00

(52) U.S. Cl. 514/57

(57) ABSTRACT

An injectable medicinal preparation is including ethanol and at least one at least partially X-ray opaque, fat-soluble compound, characterized in that it further comprises an ethanol-soluble compound which, when dissolved, has a viscosity ranging from 10 to 700 cP and preferably from 90 to 350 cP at 25° C., so as to provide an embolizing effect-inducing gel, and, in that the ethanol has a purity ranging from 70 to 99% vol., preferably from 90 to 99% vol., so as to provide a sclerosing effect. The preparation is particularly useful for treating venous malformations such as venous angiomas.

This application is a divisional application of Application No. 12/088,704 filed Jul. 9, 2008, which is a National Phase of PCT/FR2006/002213 filed Sep. 28, 2006, which claims the priority of French Application No. 05/09978 filed Sep. 30, 2005. The entire contents of each of the above-identified applications are hereby incorporated by reference.

### BACKGROUND OF THE INVENTION

The present invention relates to an injectable viscous medicinal preparation comprising ethanol and an X-ray opaque, fat-soluble compound. It is more particularly, but not exclusively, useful for treating venous malformations such as venous angiomas.

### DESCRIPTION OF THE RELATED ART

Generally speaking, venous malformations are characterized by bluish, flabby, compressible tumefactions that do swell in inclined position or by physical exertion. They may be more or less voluminous and be located in difficult to access areas or in deteriorating areas for which a surgical ablation is sometimes cumbersome or impracticable.

Pure ethanol was proposed to treat such malformations using the percutaneous route by sclerosing the malformation. Its efficiency is indisputable but there is a significant safety hazard when handling the same for the following reasons:

the sclerosing agent is in a liquid form and its diffusion beyond the pathological area is only partially controlled by carefully checking the correct positioning of the injection needle in the malformation based on a previous series of angiograms,

it is not radio-opaque and the areas that are attained by the sclerosing agent cannot be controlled neither during nor after the injection,

rare, but severe general and cardiac toxicity cases have been described that were directly associated with ethanol injection into venous malformations.

To improve pure ethanol safety and efficiency, it has been proposed to use a mixture of ethanol and a compound to provide a viscous preparation such as ethylcellulose, dextrin or a derivative thereof. Such mixture produces a gel, the two main advantages of which are as follows:
it is a gel to be injected into the venous malformation using a needle and the gel diffusion through undesirable areas is significantly restrained as compared to the same situation with pure ethanol,
the gel, because of its physical nature, remains close to the injection site and causes a much stronger sclerosing effect. The amount required for obtaining the same result is highly decreased as compared to the pure ethanol, thus lessening the risk of general toxicity.

However, this gel suffers from two significant drawbacks:
it is non radio-opaque,
a spontaneous flocculation of the sclerosing mixture (ethanol and ethylcellulose) does occur upon contacting the water-soluble contrast media traditionally used in vascular radiology, which makes it difficult for the gel to progress into the injection needle and even impossible to progress into a catheter.

### SUMMARY OF THE INVENTION

It is an object of the invention to compensate for such drawbacks.

An injectable medicinal preparation is provided, that including ethanol and at least one at least partially X-ray opaque, fat-soluble compound.

This preparation is characterized in that it further includes an ethanol-soluble compound which, when dissolved, has a viscosity ranging from 10 to 700 cP and preferably from 90 to 350 cP at 25° C. so as to provide an embolizing effect-inducing gel and, in that the ethanol has a purity ranging from 70 to 99% vol., preferably from 90 to 99% vol., so as to provide a sclerosing effect.

It should be noted that “cP” is a dynamic viscosity measuring unit, i.e. the centipoise, which corresponds to 10⁻³ Pascal second (Pa.s).

Of course, said compound which, when dissolved in ethanol, has a viscosity ranging from 10 to 700 cP and preferably from 90 to 350 cP at 25° C. does include at least one at least partially X-ray opaque, fat-soluble compound. These compounds are inert toward each other.

Active agents may be incorporated into the preparation.

More precisely, this preparation may be composed of an emulsion based on a viscous phase comprising at least the ethanol and a fat-soluble phase comprising at least said at least partially X-ray opaque, fat-soluble compound.

The preparation may includes from 10% to 50% vol. of said at least partially X-ray opaque, fat-soluble compound.

Advantageously, the more of numerous fine particles there are in the emulsion, the more homogeneous the mixture and the easier the subsequent mixture tracing will be after injection.

Said emulsion may be prepared by incorporating said at least partially X-ray opaque, fat-soluble compound either at the end of the production of the preparation, or immediately prior to the operation.

The at least partially X-ray opaque, fat-soluble compound may be a non metallic compound.

Such non metallic compound may be a halogenated fatty acid ester such as a iodine fatty acid ethyl ester.

Advantageously, in the event of venous malformations that are near to the skin and often treated mainly for aesthetic reasons, a non metallic compound will enable to avoid any black skin staining such as would occurred with metallic compounds.

Said compound having, when dissolved, a viscosity ranging from 10 to 700 cP and preferably from 90 to 350 cP at 25° C. may be selected so as to present the following properties:
being biocompatible, because of the injectable nature of the preparation,
having such a thickening power sufficient to increase the mixture viscosity, even in a small amount,
having in situ biodegradable derivatives so as to avoid any surgical resection of the treated area,
being soluble in cold ethanol so as to obtain a homogeneous preparation,
having very few, if any, local and/or systemic toxic effect(s), so as not to impair the preparation safety and optionally coming as a powder rather than in a liquid form so as not to dilute ethanol.

Said compound having a viscosity ranging from 10 to 700 cP and preferably from 90 to 350 cP at 25° C. may be ethylcellulose.

Ethylcellulose may be present in an amount varying from 0.5 to 30% and preferably from 5 to 18% by weight as related to the preparation total weight.

Therefore, the preparation of the invention does satisfy to the major required criteria which encompass the safety in use during all the process phases and the ability to perform a stable and selective sclerosis.

### BRIEF DESCRIPTION OF THE DRAWING FIGURES

Hereafter, a particular embodiment of the present invention will be described to be considered as a non limitative illustration referring to the accompanying drawings wherein:

FIG. 1 shows the viscosity values as expressed in centipoises depending on concentration as expressed in ethylcellulose weight percent based on the total weight;

FIG. 2 shows the viscosity values as expressed in centipoises depending on the temperature in degrees Celsius.

### DETAILED DESCRIPTION

### Making the Preparation

Several preparations with various ethylcellulose concentrations (Aqualon 100 NF ®, Hercules) were made by dissolving respectively 0.15, 0.45 and 0.75 g in 15 mL ethanol with a 70-99% vol. purity and preferably a 95% purity (d=0.8) i.e. 1.22, 3.61 and 5.88% by weight as related to the preparation total weight. The preparation with the highest ethylcellulose concentration was selected. A 2.5% loss during the distribution into flasks was considered, that is to say for forty flasks, 205 mL Cologne spirit (vol. %) and 10.25 g ethylcellulose. More generally, ethylcellulose is present in an amount ranging from 5 to 18%, preferably of 5.88% by weight as related to the preparation total weight.

In accordance with the good practices (Bonnes Pratiques de Fabrication—1998), the preparation included three steps: the gel preparation, the aseptic distribution and the sterilization of the product in the final packaging. In a first step, the excipient (ethylcellulose) was hot-blended with ethanol in a sterile ground neck flask, under magnetic stirring and to reflux until complete dissolution. The whole was left under stirring to reflux for 15 minutes, then under stirring until it was cold, so as to allow alcohol to recondensate in the flask. Reconditioning did then occur under a horizontal laminar air flow hood in 5 mL capacity, sterile sealed flasks (Bioblock 42065). At last, as recommended by the European Pharmacopoeia, the flasks were sterilised in autoclave under saturated steam, at 121 ° C. for twenty minutes.

The last step for making the preparation did consist in adding a fat-soluble opacifying substance such as an iodine fatty acid ethyl ester (Laboratoire Guerbet, France) with varying amounts to obtain a good opacity, for example of about 10% to 50% vol. of the total volume.

As this substance (fat-soluble phase) is not miscible with the mixture of ethanol and ethylcellulose (viscous phase), stirring the two phases did result in an emulsion which particle size depends on the applied stirring. It should be noted that the finest the particles, the more homogeneous the injected mixture, thus improving the tracing quality of the injection in proportion.

This final addition may be effected in the syringe prior to injection for example.

Moreover, this substance being inert and used in small amounts, it does not significantly modify the results given hereunder of tests performed on the preparation prior to being added.

### Tests

The preparation conformance was checked by sterility, chemical and physicochemical tests.

As recommended by the European Pharmacopoeia, the possible contaminant occurrence was controlled by seeding 4 mL of the preparation in 250 mL trypticase soy broth for aerobic germs, using thioglycolate for anaerobic germs and a Sabouraud medium for yeasts. The sterility test results did confirm the absence of contaminants in the preparation.

The alcohol content was determined after sample dilution and incorporation of the internal standard, propanol-1, by gas chromatography using a flame ionization detector. Separation was effected on a Porapak Q column (80-100 mesh, 3 m-long) with nitrogen as a carrier gas (1.2 bar) on a Delsi DN200 apparatus. As a result, the alcohol dosing did provide 802 g. L⁻¹.

The viscosity additive specific dosing was not effected but its concentration was evaluated using the dry solid matter method, which consists in evaporating ethanol in a 110° C. temperature-regulated vessel to a constant weight of the sample. The dry solid matter method made it possible to correlate the ethylcellulose theoretical concentration with that measured in the experiment i.e. 5.88% by weight of the sample total weight.

The preparation viscosity was measured by means of a capillary viscometer, also called Baumé viscometer (Prolabo). Several measurement series were performed at various temperatures and with various concentrations by thickening the preparation.

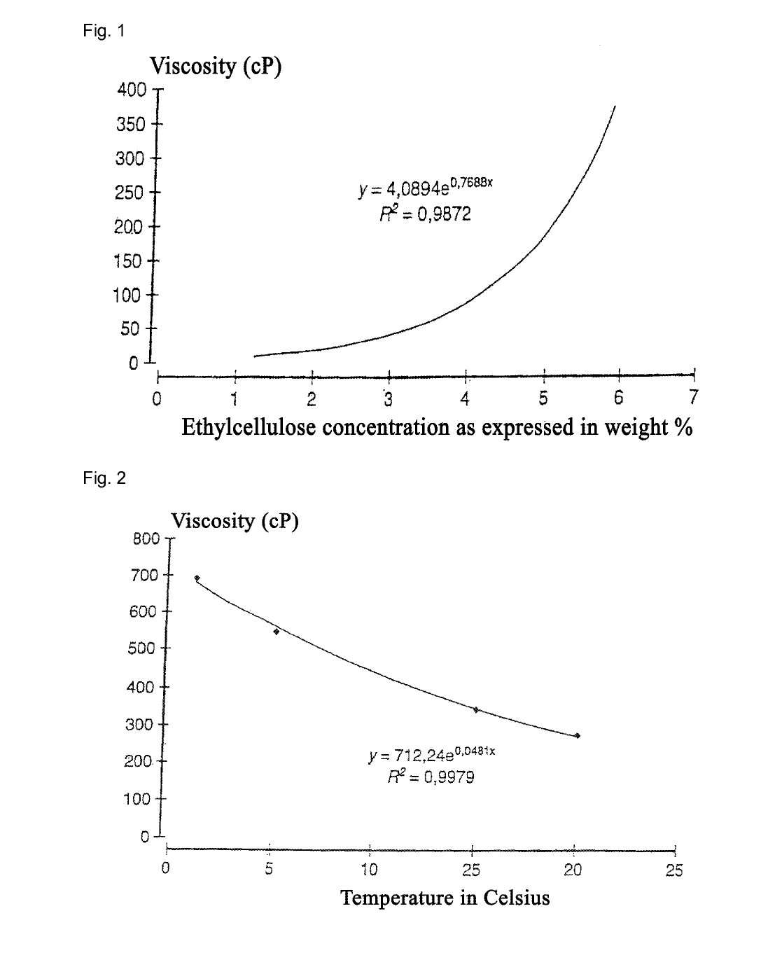

The viscosity measurements showed that at a constant temperature, the preparation viscosity and the ethylcellulose content (FIG. 1) increased exponentially. On the contrary, it still exponentially decreased with increasing temperature (FIG. 2).

To conclude, the physicochemical stability analysis was performed by measuring the evolution with the time of the parameters which define the preparation, that is to say the viscosity, as well as the ethanol and viscosity agent amounts. The measurements were done on day one (D1), and then repeated after eight days (D8), after fifteen days (D15) and after thirty days (D30).

The results are given in Table 1. Coefficients of variation lower than 3% demonstrate the mixture stability until D30, which will enable to determine the expiry date for the preparation.

TABLE I
Physicochemical parameters with respect to time

| | | | | | | | |
|---|---|---|---|---|---|---|---|
| | | | | | Dry matter | DM/Wₛₐₘₚₗₑ | |
| | Cₑₜₕₐₙₒₗ | Vₛₐₘₚₗₑ | Wₛₐₘₚₗₑ | dₛₐₘₚₗₑ | (DM) | ratio | Viscosity |
| Time | (g · L⁻¹) | (mL) | (g) | (g · mL⁻¹) | (g) | (%) | (cp)* |

| | | | | | | | |
|---|---|---|---|---|---|---|---|
| D1 | 784 | 2 | 1.650 | 0.825 | 0.101 | 6.10 | 320 |
| D8 | 821 | 2 | 1.720 | 0.850 | 0.103 | 5.97 | 339.5 |
| D15 | 783 | 2 | 1.610 | 0.800 | 0.097 | 6.00 | — |
| D30 | 820 | 2 | 1.670 | 0.830 | 0.099 | 5.92 | 332 |
| Average | 802 | — | 1.663 | 0.826 | 0.100 | 5.998 | 330.5 |
| Standard | 21.370 | — | 0.046 | 0.021 | 0.002 | 0.076 | 9.836 |
| deviation | | | | | | | |
| Coefficient | 2.665 | — | 2.751 | 2.489 | 2.458 | 1.265 | 2.976 |
| of variation | | | | | | | |

*(cp): centipoise

1. A method for the treatment of venous malformations comprising a step of administering, to a patient in need thereof, an injectable medicinal preparation comprising:
(1) an ethanol-soluble compound which, when dissolved, has a viscosity ranging from 10 to 700 cp at 25° C., so as to provide an embolizing effect-inducing gel;
(2) ethanol having, when used for obtaining said medicinal preparation, a purity ranging from 70% to 99% (v/v), so as to provide a sclerosing effect; and
(3) an halogenated fatty acid ester compound,
wherein the injectable medicinal preparation consists of an emulsion comprising (i) a viscous phase comprising at least ethanol and ethyl cellulose and (ii) a fat-soluble phase comprising the halogenated fatty acid ester compound.

2. The method according to claim 1, wherein said ethanol-soluble compound has, when dissolved, a viscosity ranging from 90 to 350 cp at 25° C.

3. The method according to claim 1, wherein said ethanol-soluble compound consists of ethyl cellulose.

4. The method according to claim 3, wherein ethyl cellulose is present in said medicinal preparation in an amount ranging from 0.5% to 30% by weight, based on a total weight of said medicinal preparation.

5. The method according to claim 3, wherein ethyl cellulose is present in said medicinal preparation in an amount ranging from 5% to 18% by weight, based on a total weight of said medicinal preparation.

6. The method according to claim 1, wherein the ethanol has a purity ranging from 90% to 99% (v/v).

7. The method according to claim 1, wherein said halogenated fatty acid ester compound is present in said medicinal preparation in an amount ranging from 10% to 50% (v/v).

8. The method according to claim 1, wherein said halogenated fatty acid ester compound is an iodine fatty acid ester compound.

9. The method according to claim 1, wherein the ethanol has a purity from 90% to 99% (v/v).

10. The method according to claim 1, wherein said medicinal preparation is administered by a percutaneous route.

11. The method according to claim 1, wherein said venous malformations are venous angiomas.
